(19) United States
(12) Patent Application Publication
(10) Pub. No.: US 2025/0154493 A1
(43) Pub. Date: May 15, 2025

(54) METHOD FOR ISOLATING NUCLEIC ACIDS BY A NOVEL HEMP MATRIX

(71) Applicant: TECHNOCULTURE RESEARCH PRIVATE LIMITED, Patna (IN)

(72) Inventors: Afreen Khanam, Sitamarhi (IN); Sana Ul Fatma, Gaya (IN); . SATYAM, Patna (IN)

(21) Appl. No.: 18/947,119

(22) Filed: Nov. 14, 2024

(30) Foreign Application Priority Data
Nov. 15, 2023 (IN) 202331078034

(51) Int. Cl. C12N 15/10

(52) U.S. Cl. CPC C12N 15/1006

(57) ABSTRACT

The present invention provides a method for nucleic acid extraction and purification from a biological sample by an alkali-cottonized hemp matrix that may be scaled up for industrial production of a novel alkali-cottonized hemp matrix. The methods/matrix as disclosed in the present invention is economically effective over the existing methods and uses the largely available hemp fiber for the process. The extraction achieved by the novel matrix of the present invention achieved better nucleic acid extraction over the existing methods/matrices.

### FIELD OF INVENTION

The invention relates to the field of bio-medical technology, particularly to a novel hemp matrix for nucleic acid purification and isolation.

### BACKGROUND

Hemp or Cannabis sativa is a naturally occurring fiber that has been more recently cultivated for textile use. The plant has a low requirement for nutrients, soil quality, and specific climatic conditions and can be easily cultivated. Several desirable qualities of hemp such as hygroscopicity, high absorbency, good thermal and electrostatic properties, protection against UV radiation, and lack of any allergenic properties make it a fiber used widely in the textile industry. One of the important uses of hemp fiber is as a raw material for the production of environmentally friendly clothes. Hemp is blended with cotton and chemical fibers, or fibrous pulp, and composite materials for its intended use in the textile industry.

Hemp is composed of several fibers, it has hemicellulose in amorphous form present as a part of cell-wall constituent, in the spaces between the fiber, in both the primary and the secondary wall. The fibers in hemp are held together by a pectinous gum. The pectin is primarily present as a component of the middle lamellae and the primary wall. The pectins of the middle lamellae are encrusted with lignin, which makes it difficult to split the bundles into fibers. The lignin, located in the middle lamella and the secondary wall, is responsible for the rigidity of the cell wall. Each elementary fiber can be considered as a network of ultrafine cellulose fibrils embedded in a matrix of hemicelluloses and lignin. The main challenge in preparing hemp fibers for textile applications is to remove these non-cellulosic substances without damage to the fiber cellulose.

The investigation of nucleic acids (NAs) is of paramount importance in comprehending fundamental life processes. DNA, characterized as a double-stranded, helical structure and commonly denoted as the “molecule of life,” is the carrier of genetic information essential for organisms' growth, development, and functioning. Conversely, RNA, a single-stranded molecule, plays a pivotal intermediary role, facilitating communication between DNA and protein synthesis processes. In the realm of molecular biology and diverse biological experiments, the extraction and purification of NAs constitute fundamental procedures to probe genetic information, gene expression patterns, and molecular interactions.

The first isolation of nucleic acids was carried out by Miescher and Altmann in the second half of the nineteenth century (Miescher, Friedrich (1871). The basic approach to extraction and purification of nuclear DNA from mammalian cells was developed about three decades ago (N. Blin, D. W. Stafford (1976). One of the earliest DNA purification methods for forensic samples was the use of phenol/chloroform extraction (D. M. Wallace (1987). The use of silica beads for DNA isolation has been a standard technique for over a quarter century, with the initial protocols based on the binding of DNA to silica in the presence of chaotropic agents such as sodium iodide (B. Vogelstein et al., (1979), silica matrices/non-chaotropic agents. Similarly, the extraction and purification process of the nucleic acid has been carried out by magnetic beads, ion exchange, Chelex, and partially automated DNA purification.

Over time, numerous extraction methodologies have been devised, prioritizing the optimization of isolated nucleic acids' efficiency, yield, and purity. The incomplete or partial DNA recovery, carryover of inhibitors impacting downstream processes, and risk of high molecular weight DNA shearing have been observed with the techniques used currently. Additionally, limitations in suitability for all sample types and the relatively high cost of silica-based column extraction are recognized. Environmental concerns arise, necessitating efforts for proper recycling or disposal of used columns. The potential hazards of silica dust and waste from silica extraction industries are significant considerations. Moreover, improper disposal of silica could lead to its breakdown into microplastic particles, posing a risk of water body contamination and harm to marine life. Alternative solution-based methods are notably laborious, and time-consuming, and necessitate the expertise of skilled laboratory personnel for proficient equipment handling.

Several states of art have tried to address the existing limitations of optimization of the process of extraction of isolated nucleic acids by overcoming certain of the above-stated limitations.

U.S. Pat. No. 10,745,686B2 published on 8 Jan. 2016 discloses a method for isolating DNA molecules having a size above a certain cut-off value from a DNA-containing sample, by contacting the sample with a binding buffer that comprises a chaotropic salt and a buffering agent to provide a binding mixture and binding DNA molecules having a size above the cut-off value to a binding matrix which has a silicon-containing surface, wherein the cut-off value is determined by the pH value of the binding mixture, separating the bound DNA from the remaining sample, washing the bound DNA, and eluting the bound DNA from the binding matrix. The binding matrix provides a silica surface for DNA binding and is a column. As discussed above, using a silica gel column results in challenges such as the high cost of the column, and the environmental hazards of disposing of the column.

JP5112064B2, published on 10 Jan. 2008, discloses a method for removing contaminants from a nucleic acid-containing sample, wherein the contaminants or inhibitors that inhibit amplification or hybridization of nucleic acids in the sample, or enzymes by adding a surfactant, such as sodium chloride, ammonium sulfate, potassium acetate, and sodium acetate, isolating nucleic acids and residual contaminants and inhibitors from the reaction mixture in the supernatant, and contacting the nucleic acid supernatant with an aggregating agent to further remove contaminants or inhibitors from the supernatant, wherein the aggregating agent are ammonium aluminum sulfate, ammonium sulfate dodecahydrate, ammonium aluminum sulfate.

The existing method of nucleic acid extraction however involves several complicated steps and is still not economically viable. Considering the paramount importance of this method and the wide use of this method in the medical industry, better methods are still required. Further, the available methods generate environmentally hazardous waste that is difficult to dispose of. A simple process that addresses the above-mentioned issue and makes the nucleic acid extraction and purification method cheap and affordable is still desirable.

Hemp fiber represents an innovative matrix for nucleic acid extraction, introducing novelty to diagnostic applications. Renowned in industries like composites and textiles, hemp's remarkable versatility finds a new sustainable role in diagnostics. Compared to traditional cotton, it offers revolutionary attributes: biocompatibility, UV resistance, unmatched tensile strength, temperature resilience, and a 20% higher adsorption capacity. These features position hemp as a groundbreaking diagnostic material. Its minimal water requirement, extended land utility, and natural resistance to pesticides further enhance its sustainability, making hemp a pioneering, eco-friendly choice for diagnostics. With the ability to produce 3.5 tons per acre and requiring only 500 Liters of water for cultivating 1 kg of hemp whereas cotton yields merely 2000 pounds per acre and takes 20000 Liters for 1 kg of cotton lint, hemp surpasses cotton in both efficiency and environmental responsibility, promising to reshape diagnostic procedures. This invention promises to redefine diagnostics, enabling more efficient, environmentally responsible, and sustainable diagnostic procedures with far-reaching implications.

### OBJECT OF THE INVENTION

The primary objective of the invention is to derive a matrix, a method for nucleic acid extraction and purification that is simple, economical, and more viable than existing methods.

Another objective of the present invention is to provide a method that is environmentally friendly and employs a biodegradable matrix.

Still, another objective of the present invention is to design a more efficient matrix for nucleic acid binding and purification than the existing matrix used for nucleic acid purification in state-of-the-art methods.

Still another object of the present invention is to employ a cheap, biodegradable, and waste material for matrix for use in nucleic acid binding, extraction, and purification process.

Another objective of the present invention is to generate and demonstrate industrial-grade large-scale pure biological macromolecules including protein, lipids nucleic acid purification, and extraction methods using a cost-effective purification matrix.

Yet another objective of the present invention is to demonstrate the cost-effective purification matrix production method process.

### SUMMARY OF THE INVENTION

The present invention provides a method for nucleic acid extraction and purification from a biological sample by loading a lysate sample to a column containing an alkali-cottonized hemp matrix followed by washing and eluting the sample-bound matrix.

In one embodiment the alkali cottonized hemp matrix may be obtained by the treatment of monovalent alkali comprising group 1A cation and/or divalent alkali 2A cation group or a combination thereof. The monovalent alkali (1A cation group) may be selected from the group comprising NaOH, KOH, LiOH, CsOH, RbOH, and other equivalent/similar monovalent hydroxides. The preferred monovalent alkali are NaOH and KOH. The divalent alkali (2A cation group) may be selected from the group comprising Mg(OH)₂, Ca(OH)₂, Ba(OH)₂, Ra(OH)₂, Be(OH)₂, and other equivalent/similar divalent hydroxides. The preferred divalent alkali are Mg(OH)₂ and Ca(OH)₂.

In one embodiment the alkali cottonized hemp matrix may be obtained by the cottonized hemp treating with a combination and/or sequential treatment of both monovalent (1A cation group), and divalent alkali (2A cation group), which confers on the surface of the treated hemp matrix both monovalent or divalent cation enabling the matrix to capture of biological macromolecules preferably nucleic acid.

In one embodiment the lysate sample is prepared by adding the lysis buffer with proteinase K. The lysis buffer may comprise surfactants and buffering agents, wherein the surfactant is selected from a group comprising guanidine thiocyanate, guanidine hydrochloride, SDS, CTAB, triton x, Tween 20, with concentrations ranging from 0.001 mM to 0.5 mM. The concentration of other reagents in the buffer may vary from 2 mM to IM. The preferred lysis buffer may comprise SDS, in the ranges from 3% to 12% preferably 7% to 10% with Tris EDTA 100 mM-200 mM (TE) and maintained at a temperature of 40-60° Celsius.

The washing step may have two or more washes, the first wash is carried out with a washing buffer comprising water-soluble alcohol, chelating agent, and salt. The water-soluble alcohol may be selected from a group comprising methanol, ethanol, n-propyl alcohol, isopropyl alcohol, and t-butyl alcohol preferably ethanol. The concentration may range from 50-90 percent (v/v) preferably 70% (v/v) ethanol. The salt may be selected from a group comprising MgCl₂, KCl, NaCl, and CaCl₂. and/or similar/equivalent salts of group 1A cation and group 2A cation, preferably MgCl₂. The concentration may range from 100-200 mM. The chelating agent may be selected from a group comprising EDTA, EGTA, EDDS, MGDA, IDS, polyaspartic acid, GLDA, BAPTA, and citric acid. In a preferred embodiment, the chelating agent is EDTA. and the second wash buffer may preferably comprise 50-90 percent ethanol.

In one embodiment, the optimized elution may be carried out at 40 to 60° C. and by an agent selected from a group comprising water, distilled water, diethyl pyrocarbonate treated water, Milli-Q water, reverse osmosis purified water (RO water), Nuclease free water. The samples may be processed prior to the nucleic acid/other biological macromolecule extraction such as by preheating and maintaining at a pH of 5-14, preferably pH 6-10.

The alkali-cottonized matrix is obtained by treating a cottonized hemp fiber with 0.1 M to 13 M alkali solution in deionized water followed by drying the hemp fibers for 30 min-48 hrs at 20-70° C., followed by washing the hemp fibers 5-6 times, wherein the pH of the fiber is maintained at 6-7 and drying the hemp fibers for 30 min-48 hrs at 40-60° C. and shredding and felting the hemp fibers. In the method disclosed the washing step is followed by air drying. The shredding is employed to separate the sticky/clogged hemp fiber into pieces with optimum sizes and increases the binding efficiency and the felting interlocks the fibers and increases the binding efficiency.

In one embodiment an industrial system of extraction and purification of biological macromolecules from a sample is disclosed encompassing an Autoclavable reagent storage tank [104] configured to collect lysed/raw samples, wherein the said incubator bio-mixer [104] is temperature-, humidity-, and pressure-controlled, Incubator bio-mixer [106] configured to receive sample and lysate reagent via pressure pumps and regulators (P1, P2, P3, P4, . . . ) at a controlled flow rate from the individual, autoclavable reagent storage tanks [104]; Matrix column chamber [110] comprising HEMP matrix column [102] connected to a Processing reagent chambers [112] comprising alkalization chambers configured to treat the cottonized hemp fiber with 5-50% alkali solution in deionized water followed by drying the hemp fibers for 30 min-48 hrs at 20-70° C., via pumps (P6, P7, P8, P9, P10, . . . . Pn); Felting and Shredding Units configured to separate clogged cottonized hemp fibers into separate fibrous structures and interlocking of cottonized hemp fibers respectively. Automated mixer chamber [114] placed downstream the matrix column chamber [110] configured to incubate and processing of samples, wherein the automated mixer chamber [114] is connected with reagent chambers via pumping (P12, P13, . . . . Pn) for sample purification and extraction process; Optionally also comprising a solid-state purification column [116] for ultra purification; and closed inert vials or sterilized sample containers [118] for storage of the processed samples.

The system may also comprise washing chambers and drying chambers, wherein the washing chambers are configured to wash hemp fibers 5-6 times, wherein the pH of the fiber is maintained at 6-7 and the drying chambers are configured to completely dry the cottonized hemp fibers.

In yet another embodiment a method of obtaining alkali-cottonized matrix by the system as described is disclosed, comprising treating a cottonized hemp fiber with 0.1 M to 1 M alkali solution in deionized water followed by drying the hemp fibers for 30 min-48 hrs at 20-70° C. in alkalization chamber, conveying the alkalized hemp to the washing chamber configured to wash the hemp fibers for 5-6 times, wherein the pH of the fiber is maintained at 6-7; conveying the washed hemp to the drying chamber and to completely dry the fibers until fully dry and moisture-free; and conveying the moisture-free alkali-cottonized hemp to the shredding and felting section.

The method and the novel matrix of the present invention under the present disclosure hold several advantages over currently available methods/matrices. With the aim of providing a better insight into the mechanism and advantages of the invention, a few examples of practical representations thereof are described further in this document, with reference to the accompanying drawings.

### BRIEF DESCRIPTION OF FIGURES

The above-mentioned objectives and descriptions, features, aspects, and advantages of the present invention will become better understood when the following detailed description is read with reference to the accompanying figures in which like characters represent like parts throughout the figures, wherein: FIG. 1 depicts the binding mechanism of monovalent embodiment where monovalent from Na+ ions onto the innovative hemp matrix post-treatment with alkali. The variables of fiber binding hemp surface with DNA and their result according to chemical bonding are as follows.

FIG. 1(a) depicts cottonized hemp fiber has a neutral charge on the surface which does not effectively bind to the DNA.

FIG. 1(b) & (c) depict alkaline treatment given to cottonised hemp fiber (alkali-cottonised treated hemp) alkali chemical reaction to form a novel fiber which gave a positive charge on the surface which is not neutralized post alkali treatment. The alkali cottonised hemp matrix has a positive charge present on the surface which binds with the DNA/RNA during the nucleic acid extraction. During alkaline treatment on the hemp, monovalent alkali in a single treatment reacts with the hydroxyl group of (—OH) of the fiber and produces water molecules (H—OH) which are consequently removed from the fiber. In between the reaction, from monovalent embodiment group IA cations bind with the O (oxygen) on the fiber surface and it gives a positive surface on the fiber. The positive charge of the alkyl-treated hemp fiber allows for strong electrostatic interactions with negatively charged nucleic acid molecules, resulting in highly efficient nucleic acid capture without the presence of any chaotropic bridge.

FIG. 2 depicts the alkylation of hemp fiber. 101 shows cottonized hemp, and 102 and 103 shows cottonized hemp post-treatment with alkali. 104 is drying of treated hemp in an oven. 105 is washing treated hemp with dH20. 106 is storing of the treated hemp. 107 is drying treated hemp in the oven. 108 involves shredding of the treated fibers of hemp. 109-111 shows the packing of treated hemp fiber in the spin column, in an embodiment of the present invention;

FIG. 3 depicts the preferable industrial process flow diagram of large-scale alkyl cottonised hemp matrix fabrication in an embodiment of the present invention;

FIG. 4 depicts a schematic representation of the innovative method of extracting nucleic acids from a treated hemp matrix in an embodiment of the present invention;

FIG. 5(A) & (B) depicts an analysis of the DNA and RNA product with the samples L1 cotton, L2 cottonized hemp, L3 Novel hemp matrix and L4 represents the Blotting Paper extracted DNA band (i) in an agarose gel; (ii) and respective densitometer trace pattern (electrophoretogram) images of the obtained DNA and RNA products.

FIG. 6 depicts nucleic acid extraction using alkyl-treated hemp fiber further amplified by PCR. L1 Molecular weight marker, L2 PCR product of DNA extracted from alkali cottonised hemp and L3 cDNA of RNA product amplified using PCR, L4 Non-template control (NTC);

FIG. 7 depicts FTIR results before (bare hemp) and after alkali treatment (treated hemp), revealing structural changes on the fiber surface and implications for fiber-matrix interface bonding. The baseline spectrum reveals the presence of distinctive peaks at 1000 cm⁻¹-1800 cm⁻¹ (CO—O—CO stretching) and 2850 cm⁻¹, corresponding to specific molecular vibrations in cottonized hemp fibers. These peaks differentiate the alkali treatment and cottonized hemp; and

FIG. 8 depicts an industrial process flowchart or process diagram of large-scale purification of nucleic acid extraction.

Further, skilled artisans will appreciate those elements in the figures illustrated for simplicity and may not have been necessarily drawn to scale. For example, the flow charts illustrate the method in terms of the most prominent steps involved to help improve understanding of the aspects of the present invention. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having the benefit of the description herein.

### DETAILED DESCRIPTION

To promote an understanding of the principles of the invention, reference will now be made to the embodiment illustrated in the figures and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. It will be understood by those skilled in the art that the foregoing general description and the following detailed description are exemplary and explanatory of the invention and are not intended to be restrictive thereof.

Reference throughout this specification to “an aspect” “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

In accordance with the current invention, provided herein is a Nucleic acid (NA) extraction process utilizing an alkyl-treated hemp fiber matrix. Said alkyl-treated hemp fiber matrix possesses a positive charge, thereby promoting facile DNA binding. The positive charge of the alkyl-treated hemp fiber matrix facilitates robust electrostatic interactions with negatively charged NA molecules, leading to exceptionally efficient NA capture. The present disclosure pertains to a nucleic acid extraction method employing a readily accessible and environmentally friendly approach, employing a fiber-based matrix. Said matrix comprises organic compounds, including cellulose, hemicellulose, lignin, pectin, and waxes, making it biocompatible and eco-friendly. Cellulose, a naturally occurring polysaccharide within plant cell walls, serves as a binding agent for DNA via hydrogen bonding and other interactions. The removal of other organic compounds is accomplished through an alkaline treatment process with neutralization. This process also makes the hemp matrix surface positively charged which ultimately serves as a binding agent for DNA via electrostatic interactions The rationale behind utilizing the cellulose-based matrix for nucleic acid extraction lies in its capacity for selective binding and retention of NAs. As an alternative to conventionally employed silica-based matrices commonly found in commercial extraction kits, the cellulose-based matrix, herein comprised of cottonized hemp fiber, has been chosen for its ease of accessibility in the market and its extensive usage in diverse fields, such as the textile industry. The cottonized hemp fiber undergoes chemical treatment before being employed as matrices for NAs extraction.

The present invention relates to a novel method for chemically treating hemp fibers purchased from the market and chemically treated with an alkaline solution, to enhance their properties and suitability for various applications. The method involves the following steps:

Hemp fiber is treated with an alkali, the process is known as alkylation. Alkylation is necessary for processing the direct influence of cellulose fibrils, the degree of polymerization, and the extraction of lignin and hemicellulose compounds. This treatment not only increases the surface roughness but also increases the amount of cellulose exposed on the fiber surface. It also increases the tensile strength, hygroscopic, and lustre of the fiber.

### Step 1: Immersion of Hemp Fibers

The cottonised hemp fibers, obtained from the market, are immersed in the prepared alkali solution. The fibers are completely submerged to allow for even and effective treatment.

The hemp fibers are allowed to undergo alkali treatment for a specific period, optimizing the treatment time based on the desired properties and the fibre's characteristics. During this treatment, the alkali penetrates the fiber structure, causing chemical modifications and improving its physical and mechanical attributes.

### Step 2: Drying of Hemp Fibers

The alkylation of the fiber is followed by a drying step of the fibers in a hot air oven for 30 min to 48 hrs at 40-70° C. or dried in sunlight for 48 hr or air dry at room temp. The treated hemp fiber drying could sterilize the fiber and remove the antimicrobials. The alkylated fiber is kept in a hot air oven for sterilization destroying the microorganism and bacterial spores which is accomplished by conduction of heat. As the fiber is composed of cellulose majorly it ignites quickly therefore keep the hemp fiber at 60° C. for 24 hr.

### Step 3: Washing and Neutralization

After the appropriate treatment time, the alkali-treated hemp fibers are thoroughly washed with water to remove excess alkali and other impurities. Subsequently, the fibers are neutralized to prevent any residual alkaline effects.

After the treatment, the alkali-treated hemp fiber is washed with water (tap water/distilled water/DEPC treated water/milli Q water/RO water) which\removes the excess amount of Alkali through the fiber.

### Step 4: Drying

The chemically treated hemp fibers are then subjected to drying in a hot air oven. The temperature and duration of the drying process are carefully controlled to ensure the complete removal of moisture without causing any damage to the fibers.

The resulting chemically treated hemp fibers exhibit enhanced properties such as improved tensile strength, increased flexibility, and better resistance to microbial degradation. Additionally, the treated fibers possess improved dye affinity and can be more easily integrated into various composite materials and products.

The chemically treated hemp fiber is dried in an air/hot air oven/sunlight heat/air drying/air blow dryer for 24 hr to 48 hr releasing the excess amount of water present in the hemp fiber.

This method offers a cost-effective and efficient approach to modifying hemp fibers, expanding their potential for use in industries ranging from textiles to construction and beyond. The treated fibers improved properties open up new avenues for sustainable and eco-friendly applications, making this process a valuable contribution to the field of natural fiber modification.

In one embodiment of the present invention, an alkaline solution (1M-13M) is prepared and hemp fiber is placed into the prepared alkaline solution. The hemp fiber is completely submerged in the solution, allowing the alkaline solution to penetrate the fibers evenly. The soaking is preferably carried out for 12-48 hours, more preferably 20-36 hours, and most preferably for 24 hours. During this soaking period, the alkali reacts with the cellulose and hemicellulose components in the hemp fiber, causing chemical changes that can improve the fiber's properties, such as increased strength and flexibility.

In an embodiment, the alkali treatment may be carried out with monovalent alkali (group 1A cations) preferably NaOH or KOH. During the treatment, the alkali reacts with cottonized hemp, disrupts the hydrogen bonds between cellulose chains, and releases water as H₂O. The group 1A cations from the alkali react with the surface of the cottonized hemp and the positive charge is conferred on the surface of the matrix. The removal of hemicellulose, lignin, and pectin (cementing materials) from the fibers results in separation and enhanced exposure of hydroxyl groups on the fiber surfaces, thereby improving interfacial bonding and fiber roughness and increasing thermal stability.

After soaking is complete, the hemp fibers are removed from the alkali solution and, dried in an oven. After the dried fiber is rinsed thoroughly with DI water (5-6 times) to neutralize any remaining alkali. The rinsed fibers are dried on a tray or rack in a hot air oven. The hot air oven to an appropriate temperature (typically around 70-40 degrees Celsius, preferably 50-60 degrees Celsius) and dry the hemp fiber for 12-48 hours, preferably 24 hours. The drying process is essential to remove excess moisture from the fibers and stabilize the chemical modifications induced by the alkali treatment.

The hemp fiber is clogged post-alkylation treatment. Shredding is a process for separating the clogged fibers into separate fibrous structures. In one embodiment the dried hemp fiber is shredded by hand to maintain the length of the fiber and cause less damage to it. Felting is a process that is used for mechanical action, causing the interlocking, or matting, of fibers possessing. In one embodiment the shredded hemp fiber is felted with a needle to make it porous and fibrous compact packed into the spin column. The treated hemp fiber (novel fiber) is in a Petri plate or air-tight container.

Once the drying process is complete, you may want to conduct various tests to evaluate the effects of the alkali treatment on the hemp fiber's properties. Common tests include tensile strength testing, moisture content determination, and microscopic analysis to assess changes in the fiber's structure. Common test like FTIR analysis is done to assess organic components characterization based on treated and cottonised hemp fiber.

After the drying and analysis, the treated hemp fiber was stored in a dry and clean Petri plate to preserve its properties with a sealed film.

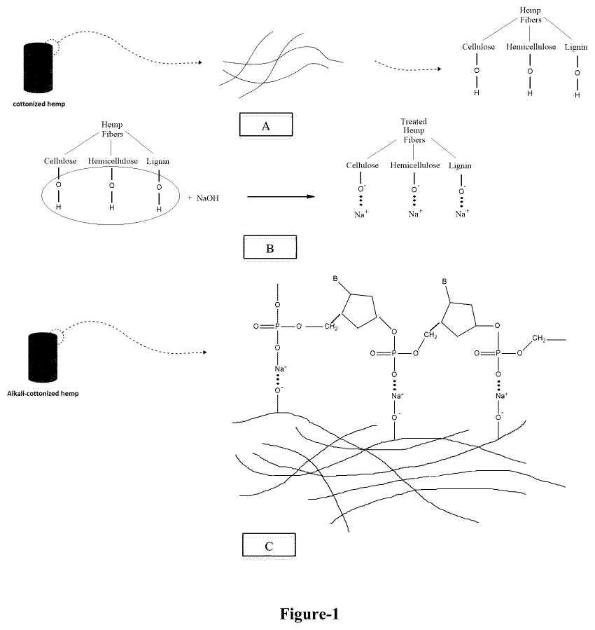

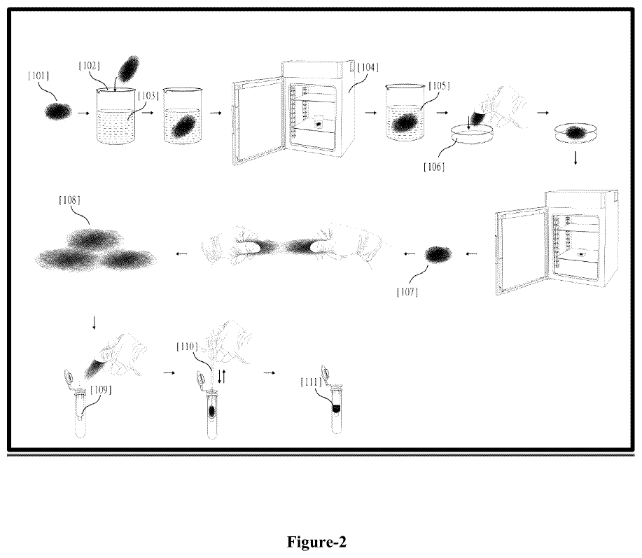

FIG. 1 depicts the binding mechanism of the monovalent treatment of Na+ ions onto the innovative hemp matrix subsequent to its treatment with an alkali solution in one embodiment of the invention. Comparative analysis between cottonized hemp and modified hemp matrix exhibits an augmented positive charge across its surface. This enhanced charge property enables the alkali cottonized hemp matrix to exhibit heightened efficacy in binding with molecules carrying a negative charge.

In one embodiment, the present invention discloses a method for the isolation of nucleic acid from a sample, the method comprises steps are follows:
i. adding a lysis buffer with proteinase K and incubating the sample;
ii. adding the ethanol to lysate the sample and transferring it into a column containing an alkali cottonised hemp matrix; and
iii. washing and eluting the matrix-bound nucleic acid.

In another embodiment, the biological sample was selected from the group of sputum, saliva, tissue, tears, stool, urea, and extract but the preferred sample selected was blood.

In another embodiment of the present procedure, the lysis buffer with the group of surfactant and buffering agent, where the surfactant is selected from a group comprising guanidine thiocyanate, guanidine hydrochloride, SDS, CTAB, triton x, Tween 20, with different concentration of ranges from 2 mM to 100 mM Guanidine-HCl, 2 mM to 100 mM guanidine thiocyanate, and other reagent ranges from the 2 mM to 1M. The preferred lysis buffer selected may comprise SDS, in the ranges from 3% to 12% but the preferred range is 7% to 10%.

In another embodiment, the lysis buffer comprises a group of buffering agents whereas the preferred buffering agent is TE buffer ranges in concentration from 2 mM to 1 M but the preferred range is 100 mM to 200 mM.

In another embodiment, the biological sample with lysis buffer heating is needed with the various ranging 40-80 degrees C. for 3-15 min, the preferred range is 56-65 degrees C. for 10 min before the ethanol addition and after elution is added to the sample.

In an embodiment, the sample containing lysis buffer spinning may be required for the lysate to be mixed properly with the reagent generally preferred equipment is a vortex.

In an embodiment, the preferred washing buffer is selected from a group comprising the ethanol with various types of salt including NaCl, Mgcl2, KCl, and others where the preferred washing buffer may have a concentration in ranges from 2 mM-1M MgCl₂, 0-200 mM MgCl₂, 50-90% ethanol or only 50-90% ethanol as washing buffer.

In another embodiment, the elution buffer may contain, Tris HCl, T.E buffer (10 mM Tris EDTA), PBS (phosphate buffered silane) containing NaCl, KCl commonly with a concentration 1× or 10× utilized to enhance the yield and the purity of nucleic acid. In a preferred embodiment, the preheated sample may be used for the elution for the extraction of nuclease-free water with a maintained pH of 6-10.

In another embodiment, various elution buffers may be incubated at 45-99° C. for complete recovery of nucleic acids. The deionized water used in buffer-making in the elution step can be replaced with DNase, RNase-free water, MilliQ water filtered water tap water, or groundwater with any chelating agent and Tris Buffer.

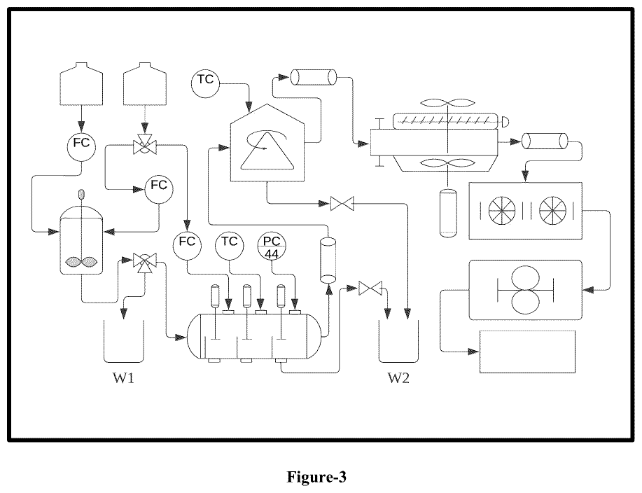

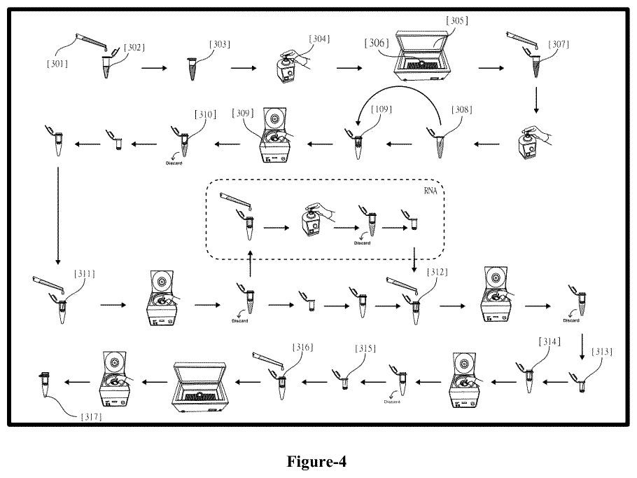

FIG. 3 depicts a preferable industrial process flow diagram of large-scale hemp matrix fabrication. The bulk of cottonised hemp fiber is conveyed to the temperature and pressure-controlled alkalization chamber through a conveyor and completely immersed in the monovalent and divalent alkali solution which is supplied through the alkali solution mixing chamber. Preferably alkali solution is prepared but not limited to 1% to 50% raw monovalent or divalent alkali and DI water is mixed inside the monovalent/divalent alkali solution mixing chamber, which is supplied to the alkalization chamber. Preferably the alkalization treatment is held up to 5 minutes to 3 days preferably but not limited to 24 hours and the temperature is maintained at 20-90 Degrees C. preferably but not limited to 60 Degrees C. The treated Hemp is washed several times like 7 to 9 times with DI water. The treated hemp is taken out from the alkalization chamber through a conveyor and conveyed to the high-speed centrifuge machine to drain out the excess amount of water. After that, the treated hemp is conveyed to the temperature-controlled drying cabinet. The drying of the treated hemp is carried out preferably for 24 h to 48 h and maintained at 20-90 Degrees C. preferably but not limited to 60 degrees C. The relative humidity is maintained at 1% to 40%, preferably, but not limited to below 20% After complete drying the treated hemp will be conveyed to a shredding machine, to make the hemp fiber puffier and to make the surface of the treated hemp fiber. After completing the shredding, the treated as well as shredded hemp is further conveyed to the felting machine to make the homogeneous and compact hemp fiber matrix.

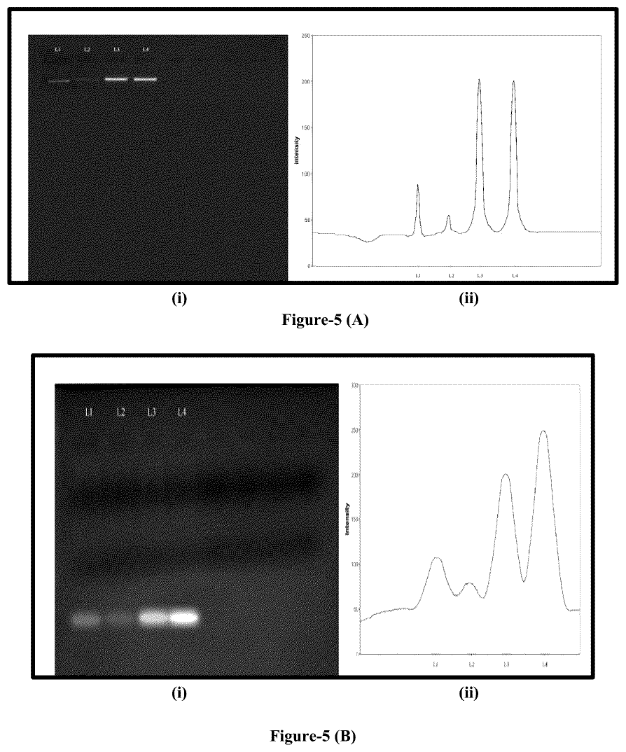

FIG. 5(A) depicts a comparison of the DNA extraction and purification obtained by L1 cotton, L2 cottonized hemp, L3 alkali-cottonized hemp matrix and L4 represents the extracted DNA. The DNA sample extracted by the novel alkali cottonized HEMP matrix was obtained as a thick gel band (L3) in an agarose gel and the graph of Fourier transform infrared spectroscopy, the highest peak of extracted and purified DNA was obtained with novel treated matrix when compared to the DNA sample extracted by the cotton and cottonized hemp matrices.

FIG. 5 (B) depicts a comparison of the RNA extracted with L1 cotton, L2 cottonized hemp, and L3 alkali-cottonized hemp matrix and L4 represents the Blotting paper. The alkali-cottonized hemp matrix shows a thick gel band (L3) and in the graph of Fourier transform infrared spectroscopy, the highest peak of extracted and purified RNA was obtained with novel treated matrix when compared to the RNA sample extracted by the cotton and cottonized hemp matrices. The experiments therefore clearly show the enhanced efficiency of the novel treated matrix, when compared to the other state-of-art matrices.

In the below table, the DNA binding efficiency has also been quantified with the general cost in USD in table format which clearly shows the cost-effectiveness is differentiated from other matrices.

| Matrix material | USD per of gram nucleic acid |
|---|---|
| 100% COTTON | 0.047 $ per gram |
| HEMP | 0.0045 $ per gram |
| ALKALI COTTONIZED HEMP | 0.025 $ per gram |
| BLOTTING PAPER | 0.16 $ per spot |
| SILICA | 2.3 $ per column |

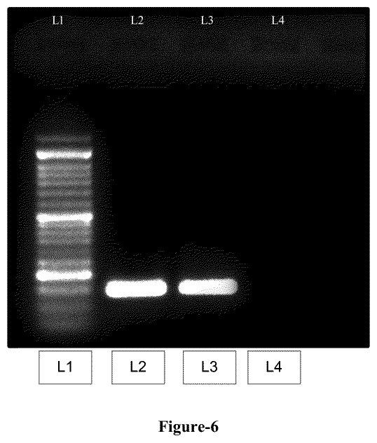

FIG. 6 clearly shows that the nucleic acid extraction using alkyl-treated hemp fiber that the PCR product of DNA extracted from alkali cottonized hemp and cDNA of RNA product amplified using PCR is of high purity, present as thick bands in lane L2 and L3.

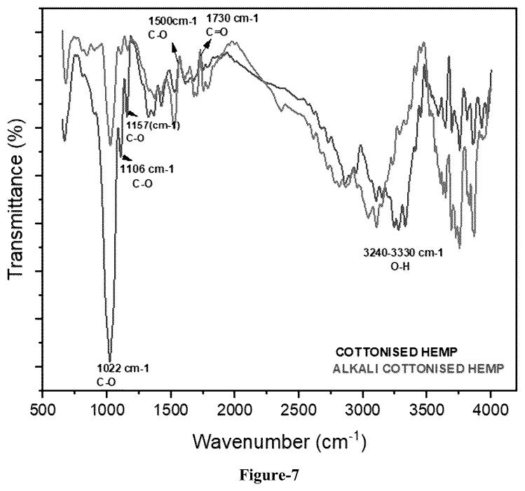

FIG. 7 depicts the FTIR analysis which is a common test done on treated and untreated hemp fiber for the clarification of functional groups present in both types of fibers. A change in the characteristic pattern of absorption bands is a change in the composition of the material or the presence of contamination. The treatment affects the fiber component showing the peaks in decreasing and removal of functional groups like acetyl, hydroxyl, and carbonyl group relation with hemicellulose, lignin, and pectin.

The graph is plotted in FIG. 7 on the basis of transmittance v/s wavelength which is compared with cottonized hemp and alkali cottonized hemp. peaks defined in grey color 1022 C—O; 1157 C—O; 1106 C—O show the presence of lignin in cottonized hemp which is shifted after treatment in alkali cottonized hemp peaks in red colour. Peaks 1500 C—O; and 1730 C—O show the presence of hemicellulose which is partially shifted in alkali cottonized hemp. Peaks 3240-3330 O—H stretch of cellulose and pectin both represent here which define the presence of partial shift of pectin and presence of cellulose in alkali cottonized hemp.

1500-1730 content C—O (carboxyl) and C═O (carbonyl) respectively which represents hemicellulose, peaks which is merged with treated hemp and cottonised hemp fiber, lower and higher peak show the hemicellulose which is the rigid material also affected by treatment.

3240-3330 content O—H (hydroxyl) group represents the pectin component in cottonized fiber after alkali treatment; this region also shows the removal of pectin to a certain extent.

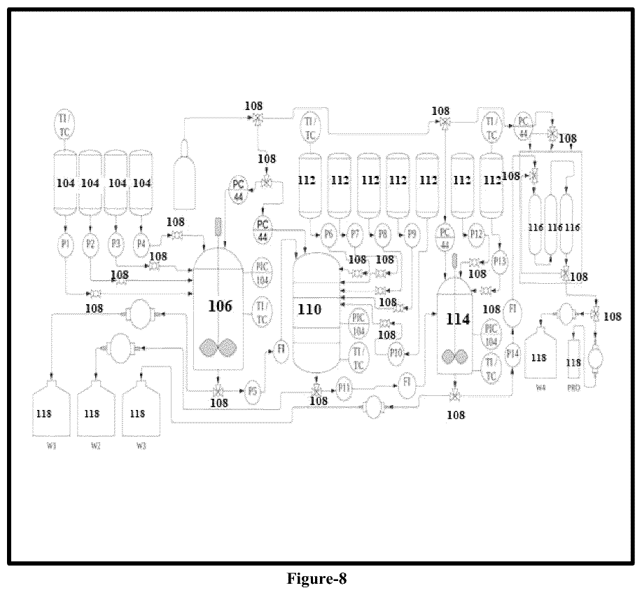

FIG. 8 shows the alkali cottonized hemp matrix column is used for collecting or capturing a large amount of nucleic acid sample by just passing the lysed or raw sample from that. The raw sample or biological sample is prepared inside a temperature, humidity, and pressure-controlled bio incubator mixer. Different pressure pumps and regulators (P1, P2, P3, P4, . . . ) control the flow rate of the raw sample or biological samples as well as the lysate reagents from the individual, autoclavable Reagent storage tanks. Some of them can be temperature, humidity, and pressure controlled. Every reagent and sample can only flow through closed steel pipes operated at each junction with individual 2-way, 3-way, 4-way, N numbers of way valves as per branching. Some of the piping required pressure, temperature, and humidity controlled by pumping the sample or reagents. The alkali cottonised hemp matrix column is placed inside the matrix column chamber which may be fully autoclavable, with temperature, humidity, and pressure controlled. This matrix column chamber can be further connected with different cleaning and processing reagent chambers with pumping (P6, P7, P8, P9, P10, . . . . Pn) and piping, which helps to further cleaning, sample purification, and extraction process. Specific purified RNA and DNA extraction methods may involve further steps. A temperature, pressure, humidity humidity-controlled fully automated mixer chamber can be placed or connected just after the matrix column chamber which can further process the nucleic acid samples to incubate and overnight processing. The temperature, pressure, humidity-humidity-controlled fully automated mixer chamber can be connected with reagent chambers which are further connected with pumping (P12, P13, . . . . Pn) and piping, which helps to purify sample purification and extraction process. The sample may pass through some solid-state purification column for ultra-purification and then finally be stored in a closed inert vial or purified sample container. Every sample processing chamber can be autoclaved and an inert gas supply is provided as well and wastes are collected in different waste chambers. The alkali cottonised hemp matrix column as well as the purification column can be replaced easily when the quality of the final output is not optimal.

In one embodiment the cottonized hemp fiber was weighed and treated with 0.1 M to 13 M NaOH/KOH in deionised water. The alkylated hemp fiber is dried in a hot air oven for 30 min, 1 hr, 6 hr, 12, 24, and 48 hours. The treated hemp fiber is washed 5-6 times with deionized water. The washed hemp fiber is then put in an open-lid petri dish and exposed to air for drying. The cottonized hemp fiber was dried in a hot air oven for 30 min, 1 hr, 6 hr, 12, 24, and 48 hours. The hemp fiber is clogged post-alkylation treatment. The dried hemp fiber is shredded by hand to maintain the length of the fiber and cause less damage to it. The shredded hemp fiber is felted with a needle to make it porous and fibrous compact packed into the spin column. The treated hemp fiber (novel fiber) in a petri plate or air-tight container.

In another embodiment, the hemp fiber could also be treated with Na2CO3 for the removal of some hard and rigid compounds like lignin which also strengthens the fiber and separates the impurities which consecutively helps in binding with nucleic acid and the extraction process.

The cottonized hemp fiber was weighed and treated with 0.1M to 13M monovalent/divalent alkali (group 1 or 2 cations) in deionized water. The alkylated hemp fiber is dried in a hot air oven for 30 min, 1 hr, 6 hr, 12, 24, and 48 hours. The treated hemp fiber is washed 5-6 times with deionized water. The washed hemp fiber is then put in an open-lid petri dish and exposed to air for drying. The cottonized hemp fiber was dried in a hot air oven for 30 min, 1 hr, 6 hr, 12, 24, and 48 hours. The cottonized hemp fiber was treated with Ca(OH) 2 for 5-6 times with deionized water. The hemp fiber is clogged post-alkylation treatment. The dried hemp fiber is shredded by hand to maintain the length of the fiber and cause less damage to it. The shredded hemp fiber is felted with a needle to make it porous and fibrous compact packed into the spin column. The treated hemp fiber (novel fiber) is in a Petri plate or air-tight container.

The cottonized hemp fiber was weighed and treated with 0.05 M to 7 M in deionized water. The alkylated hemp fiber is dried in a hot air oven for 30 min, 1 hr, 6 hr, 12, 24, and 48 hours. The treated hemp fiber is washed 5-6 times with deionized water. The washed hemp fiber is then put in an open-lid petri dish and exposed to air for drying. The cottonized hemp fiber was dried in a hot air oven for 30 min, 1 hr, 6 hr, 12, 24, and 48 hours. The dried hemp fiber is shredded by hand to maintain the length of the fiber and cause less damage to it. The shredded hemp fiber is felted with a needle to make it porous and fibrous compact packed into the spin column. The treated hemp fiber (novel fiber) is in a Petri plate or air-tight container.

In accordance with the disclosure herein, the comprehensive process of alkyl treatment is succinctly delineated in Table A, comprising a quintessential sequence of five pivotal stages, commencing with the preparation of a monovalent embodiment with single alkali treatment solution, subsequently extending to the treatment of the matrix with said solution, thence proceeding to a meticulous washing and drying regimen, culminating in the conclusive step of encapsulating the treated matrix within the confines of a specialized spin column, all as set forth herein. The DNA extraction procedures have been comprehensively elucidated and are visually represented in Table B.

For RNA extraction, it is imperative to note that all procedures shall remain identical to those delineated for DNA extraction, with the sole exception being the inclusion of a crucial step involving the incorporation of DNase enzyme subsequent to the washing process, as further detailed herein. An array of diverse matrices employed to enhance and optimize nucleic acid extraction yields has been methodically encapsulated within Table C. Likewise, the potential permutations and combinations of lysis buffer, wash buffer 1, wash buffer 2, and elution buffer have been meticulously detailed in Tables D, E, F, and G, respectively. The comprehensive compilation of experimental endeavors encompassing all conceivable combinations, undertaken to maximize nucleic acid yield, is documented in Table H for elucidation and reference. The DNA binding efficiency of the treated matrix is described in Table I.

TABLE A
Alkyl Treatment of Hemp Fiber (Cannabis sativa)

| Steps | Process |
|---|---|
| Step- 1 | A 5-50% solution of monovalent or divalent alkali solution |
| | by adding DI water. |
| Step- 2 | Commercially available bleached hemp fiber (Cannabis |
| | sativa) was dipped into that alkali solution for 30 minutes at |
| | room temperature for alkalization. |
| Step- 3 | The above step was followed by rinsing the well 3 times |
| | with DI water for 5 minutes. |
| Step- 4 | The alkyl-treated hemp was kept in a clean container and the |
| | fibers were gently spread using tweezers. The container was |
| | placed in a dry place at room temperature overnight to |
| | completely dry it (laminar airflow). |
| Step- 5 | The dry alkyl-treated hemp was packed in a spin column |
| | tightly. The process is performed under a laminar airflow. |
| | The alkyl-treated hemp spin column was stored in a sterile |
| | place. |

TABLE B
DNA Extraction Standard Protocol 1

| Steps | Description |
|---|---|
| Step -1 | A 400 μL of Lysis Solution and 20 μL of Proteinase K |
| | Solution was added to 200 μL of whole blood, and mixed |
| | thoroughly by vortex or pipetting to obtain a uniform |
| | suspension. |
| Step -2 | The sample was incubated at 56° C. for 10 min followed by |
| | a vortex for 30 sec. |
| Step -3 | 200 μL of ethanol (96-100) was added and mixed |
| Step -4 | The lysate was transferred to the novel spin column. |
| Step -5 | The column was centrifuged for 1 minute at 8000 rpm. The |
| | collection tube containing the flow-through solution was |
| | discarded. The extra column was placed into a new 2 mL |
| | collection tube. |
| Step -6 | 500 μL of wash buffer I was added and centrifuged for 1 |
| | minute at 8000 rpm. The flow-through was discarded and |
| | placed in the purification column back into the collection |
| | tube. |
| Step -7 | 500 μL of wash buffer II was added to the novel spin column |
| | purification column and centrifuge for 3 min at maximum |
| | speed (≥12000 g). |
| Step -8 | The collection tube was emptied and the column was re- |
| | spined for 3 min at maximum speed ≥. 12000 rpm. |
| Step -9 | The collection tube containing the flow-through solution is |
| | discarded and transferred to the Novel spin column to a |
| | sterile 1.5 mL microcentrifuge tube. |
| Step -10 | Elution buffer (100 μL) was added to the center of the novel |
| | spin column. The column was heated at 50-60° C. for 2-3 |
| | mins and centrifuged for 1 min at 8000 rpm. |
| Step -11 | The purification column was discarded and the purified DNA |
| | was used immediately in downstream applications or stored |
| | at −20° C. |

TABLE C
Different Matrix for DNA extraction

| Matrix ID | Matrix Details |
|---|---|
| M1 | Silica column from a thermos |
| M2 | Treated Hemp |
| M3 | Treated boiled Hemp |
| M4 | Blotting paper |

TABLE D
Different Lysis solution (L) composition

| ID | Description |
|---|---|
| L3 | 400 μL of Lysis Solution (10% SDS + 100 mM Tris-HCl + 100 |
| | mM EDTA) and 20 μLof Proteinase K Solution were added to |
| | 200 μL of whole blood, and mixed thoroughly by vortex or |
| | pipetting to obtain a uniform suspension. The sample was |
| | incubated at 56° C. for 10 min followed by vortexed for 30 sec. |
| | About 200 μL of ethanol was added (96-100%) and mixed by |
| | pipetting or vortex. |
| L4 | 400 μL of Lysis Solution (50 mM Tris-HCl, 50 mM EDTA, 2M |
| | Guanidine-HCl, 2M Urea, 10 mM Calcium Chloride, 10% |
| | TWeen-20) and 20 μL of Proteinase K Solution was added to |
| | 200 μL of whole blood, and mixed thoroughly by vortex or |
| | pipetting to obtain a uniform suspension. The sample was |
| | incubated at 56° C. for 10 min and vortexed for 30 sec. About |
| | 200 μL of ethanol was added (96-100%) and mixed by pipetting or |
| | vortex. |
| L5 | 400 μL of Lysis Solution (10% SDS + 100 mM Tris-HCl + 100 |
| | mM EDTA + 300 mM NaCl) and 20 μL of Proteinase K Solution |
| | were added to 200 μL of whole blood, and mixed thoroughly by |
| | vortex or pipetting to obtain a uniform suspension. The sample |
| | was incubated at 56° C. for 10 min and vortexed for 30 sec. About |
| | 200 μL of ethanol was added (96-100%) and mixed by pipetting |
| | or vortex. |
| L6 | 400 μL of Lysis Solution (thermo fisher std) and 20 μL of |
| | Proteinase K Solution were added to 200 μL of whole blood, and |
| | mixed thoroughly by vortex or pipetting to obtain a uniform |
| | suspension. The sample was incubated at 56° C. for 10 minutes |
| | and vortexed for 30 sec. About 200 μL of ethanol was added (96- |
| | 100%) and mixed by pipetting or vortex. |
| L7 | 200 μL of Lysis Solution (50 mM Tris-HCl, 50 mM EDTA, 2M |
| | Guanidine-HCl, 2M Urea, 10 mM Calcium Chloride, 10% |
| | TWeen-20) and 20 μL of Proteinase K Solution was added to 200 |
| | μL of whole blood, and mixed thoroughly by vortex or pipetting |
| | to obtain a uniform suspension. The sample was incubated at 56° |
| | C. for 10 minutes and vortexed for 30 sec. About 500 ul Binding |
| | buffer (1.25M NaCl with 10% PEG 8000) was added and vortexed. |
| | About 200 μL of ethanol was added (96-100%) and mixed by |
| | pipetting or vortex. |

TABLE E
Different Wash buffer - 1 (W1) solution composition

| ID | Description |
|---|---|
| W1A | Thermofisher Scientific Standard buffer. |
| W1B | 70% ethanol with 50 mM MgCl₂ |
| W1C | 2.25M NaCl with 10% PEG 8000 |
| W1D | 70% ethanol with 2 mM NaCl |
| W1E | 70% ethanol |

TABLE F
Different Wash buffer - 2 (W2) solution composition

| ID | Description |
|---|---|
| W2A | Thermofisher Scientific Standard buffer. |
| W2B | 70% ethanol |
| W2C | 2.25M NaCl with 10% PEG 8000 |
| W2D | 70% ethanol with 2 mM NaCl |

TABLE G
Different elution buffer solution (E) composition

| ID | Description |
|---|---|
| E1 | Thermofisher Scientific Standard buffer. |
| E2 | Distilled water |
| E3 | Mol Bio Diagnostics Pvt Ltd |

TABLE H
Experimental details

| SL. No. | 1 | 2 | 3 | 4 | 5 | 6 | 7 |
|---|---|---|---|---|---|---|---|
| Sample type | M1 | M2 | M3 | M2 | M2 | M2 | M2 |
| L ID | L6 | L3 | L3 | L3 | LA | L7 | L3 |
| L volume | 820 | 820 | 820 | 820 | 820 | 1120 | 820 |
| L RPM | 8000 | 10000 | 10000 | 10000 | 10000 | 10000 | 12000 |
| L Time | 60 | 120 | 120 | 120 | 120 | 120 | 120 |
| W1 ID | W1A | W1B | W1B | W1B | W1B | W1C | W1D |
| W1 RPM | 10000 | 8000 | 8000 | 8000 | 8000 | 8000 | 10000 |
| W1 volume | 500 | 500 | 500 | 500 | 500 | 500 | 500 |
| W1 Time | 60 | 60 | 60 | 60 | 60 | 60 | 60 |
| W1 wash repeat | n/a | n/a | n/a | n/a | n/a | 2 | n/a |
| W2 ID | W2A | W2B | W2B | W2B | W2B | W2C | W2D |
| W2 RPM | 14000 | 12000 | 12000 | 12000 | 12000 | 8000 | 1000 |
| W2 volume | 500 | 500 | 500 | 500 | 500 | 500 | 500 |
| W2 Time | 180 | 120 | 120 | 120 | 120 | 60 | 60 |
| W2 wash repeat | n/a | n/a | n/a | n/a | n/a | 2 | n/a |
| Drying RPM | 14000 | 14000 | 14000 | 14000 | 14000 | 1000 | 1000 |
| Drying Time | 180 | 180 | 180 | 180 | 180 | 60 | 60 |
| Drying temperature | RT | RT | RT | RT | RT | RT | RT |
| E ID | E1 | E1 | E1 | E2 | E2 | E3 | E1 |
| E RPM | 6000 | 6000 | 6000 | 6000 | 6000 | 6000 | 6000 |
| E Temp | RT | 55 | 55 | 55 | 55 | 85 | 65 |
| E time | 120 | 300 | 300 | 300 | 300 | 600 | 600 |
| E volume | 100 | 100 | 100 | 100 | 100 | 100 | 100 |
| 260/280 | 1.956 | 1.804 | 1.768 | 1.661 | 1.696 | 1.531 | 1.649 |

| SL. No. | 8 | 9 | 10 | 11 | 12 | 13 |
|---|---|---|---|---|---|---|
| Sample type | M3 | M2 | M2 | M4 | M4 | M2 |
| L ID | L3 | L6 | L3 | L6 | L6 | L3 |
| L volume | 820 | 820 | 820 | 820 | 820 | 520 |
| L RPM | 12000 | 8000 | — | 8000 | 8000 | — |
| L Time | 120 | 60 | — | 60 | 60 | — |
| W1 ID | W1E | W1A | W1B | W1A | W1E | W1B |
| W1 RPM | 10000 | 10000 | — | 10000 | 10000 | — |
| W1 volume | 500 | 500 | 500 | 500 | 500 | 800 |
| W1 Time | 60 | 60 | — | 60 | 60 | — |
| W1 wash repeat | n/a | n/a | 2 | n/a | n/a | n/a |
| W2 ID | W2B | W2A | W2B | W2A | W2B | W2B |
| W2 RPM | 1000 | 14000 | — | 14000 | 14000 | — |
| W2 volume | 500 | 500 | 500 | 500 | 500 | 800 |
| W2 Time | 60 | 180 | — | 180 | 180 | — |
| W2 wash repeat | n/a | n/a | 3 | n/a | n/a | n/a |
| Drying RPM | 1000 | 14000 | — | 14000 | 14000 | 14000 |
| Drying Time | 60 | 180 | 900 | 180 | 180 | 180 |
| Drying temperature | RT | RT | 50 | RT | RT | RT |
| E ID | E1 | E1 | E1 | E1 | E1 | E1 |
| E RPM | 6000 | 6000 | — | 6000 | 6000 | 6000 |
| E Temp | 60 | 95 | 50 | 60 | 60 | 60 |
| E time | 300 | 600 | 120 | 300 | 300 | 50 |
| E volume | 100 | 100 | 150 | 100 | 100 | 100 |
| 260/280 | 1.644 | 1.609 | 1.603 | 1.71 | 1.556 | 1.98 |

Units: All volumes are in μl, incubation times are in sec, and temperatures are in ° C.

TABLE I
DNA Binding efficiency on variable time treatment Experiment

| Total DNA before | | | Binding |
|---|---|---|---|
| Binding to Hemp | | | Effi- |
| Fiber (DNA present | Total DNA After Binding to hemp | | ciency |
| in Blood cell lysate) | (DNA present in Blood cell lysate) | | (%) |
| 7.72 ng/ul (Total | 24 hrs monovalent | 2.49 ng/ul (Total | 67.75% |
| DNA = 6330.4) | alkali Treatment | DNA = 2041 ng) | |
| 7.92 ng/ul (Total | 6 hrs monovalent | 2.89 ng/ul (Total | 63.51% |
| DNA = 6494.4 ng) | Treatment | DNA = 2369.8 ng) | |
| 7.16 ng/ul (Total | 30 min monovalent | 3.12 ng/ul (Total | 56.42% |
| DNA = 5871.2 ng) | Treatment | DNA = 2558.4 ng) | |

Isolation of RNA from Human Blood

RNA extraction procedure where 200 uL of blood is mixed with 400 uL of Lysis Buffer (10% SDS and 200 TE buffer and Dnase I) and 20 uL of Proteinase K, vortexed, and heated at 65° C. for 10 minutes. The mixture is then mixed with 200 uL of Absolute Ethanol and transferred to a spin column. The lysate is centrifuged at 5000-10,000 rpm for 30 sec to 3 minutes, and the flow-through is discarded. The samples are then first washed with 600 uL of wash buffer (70%-90% ethanol with 200 Mgcl2) and add DNase in the sample and left for 10 min the second wash is 600 uL of 70% ethanol and centrifuged at 5000-10,000 rpm for 30 sec to 3 minutes, and centrifuged again. Then the sample is sent for air drying and another round of centrifugation, and the spin column is transferred to a fresh tube. The final elution step involves adding 50-100 uL of Nuclease-free water heating the tube below 60° C. for 5 minutes, and centrifuging at 2,000-8,000 rpm for 1 minute to collect the RNA.

TABLE 1
Comparative Account of Important Aspects Involved in Isolation
of Nucleic Acid and the Property of Cellulose-Based Matrices

| | | N.A using | N.A | N.A using | N.A |
|---|---|---|---|---|---|
| | | cottonized | using Alkali | Blotting | using |
| S. No. | Property | Hemp Fiber | cottonized hemp | Paper | Cotton |
| 1. | Single protocol for DNA and RNA | No | Yes | No | No |
| 2. | RT. PCR suitable for extraction | No | Yes | Yes | Possible |
| 3. | High absorbance | Yes | Yes | Yes | Yes |
| 4. | Porous structure | Yes | Yes | Yes | Yes |
| 5 | Chemical compatibility | No | Yes | No | No |
| 6 | Ability to process different | No | Yes | Yes | Possible |
| | samples like blood, sputum, | | | | |
| | serum, saliva, tissues, etc. | | | | |
| | with minimal sample processing | | | | |
| 7 | Antimicrobial property | No | Yes | Yes | Yes |
| 8 | Biocompatibility | Yes | Yes | Yes | Yes |

### Nucleic Acid Purity

In the realm of molecular biology and biotechnology, nucleic acid purity constitutes a fundamental aspect. It pertains to the extent to which a specimen of nucleic acid, exemplified by DNA or RNA, is devoid of adulterants, unwanted alterations, or extraneous molecules. The purity of nucleic acids assumes paramount significance for various salient motives. Elevated nucleic acid purity stands as an indispensable requisite for upholding the unblemished integrity of research data. The intrusion of contaminants or impurities can precipitate inaccuracies in results and misconceptions regarding the outcomes of experiments. Across diverse biotechnological applications, encompassing PCR, sequencing, gene expression analysis, and cloning, the quality, and purity of nucleic acids wield a direct and profound influence upon the dependability and prosperity of these methodologies. Within the realm of molecular diagnostics, which leans upon nucleic acid scrutiny, there exists an exigency for exceptionally pure commencing materials. Contaminants hold the potential to instigate spurious affirmative or negative results, thereby imperiling the precision of patient diagnoses and therapeutic decisions. In the sphere of developing nucleic acid-based therapeutic modalities, spanning gene therapies and RNA-based medicaments, heightened purity assumes a non-negotiable role in ensuring safety and efficacy. The 260/280 optical density (OD) ratio emerges as a pivotal parameter during the appraisal of DNA sample purity and quality. This ratio hinges upon the absorbance magnitudes of DNA at two precise wavelengths, namely 260 nm and 280 nm, ascertained through the employment of a spectrophotometer. The significance of the 260/280 OD ratio resides in its aptitude to furnish valuable insights into the constitution and plausible contaminants existing within a DNA specimen.

TABLE 2
Comparison of DNA purity with different matrix and reagents

| | OD260/280 |
|---|---|
| Matrix and reagent details | (DNA purity) |
| Cotton + standard reagent mix | 0.7-1.5 |
| Cotton + optimized reagent mix | 1.5-1.6 |
| Cottonized hemp + standard reagents mix | 0.2-0.9 |
| Cottonized hemp + optimized reagent mix | 1.3-1.5 |
| Alkyl cottonised hemp + standard reagents mix | 1.3-1.5 |
| Alkyl cottonised matrix + optimised reagent mix | 1.7-1.8 |

Table 2 illustrates the comparison of the purity of DNA obtained using different matrices with possible combinations of reagents mix. The alkali cottonized hemp matrix along with the optimised reagents mix combination gives optimal DNA purity with OD260/280 value between 1.7 and 1.8.

An exemplary embodiment employed a wash buffer 1 comprising 70% ethanol and NaCl. This configuration resulted in DNA purity ranging from approximately 1.5 to 1.6. Likewise, the utilization of a lysis solution containing SDS at concentrations ranging from 1% to 5% yielded DNA purity levels within the range of approximately 1.2 to 1.5. A comprehensive elucidation of reagent optimization is briefly presented in Table 2 for reference and clarity.

Optimized reagent mix compromises the lysis, washing, and elution buffer where lysis may contain 10% SDS, 200 mM TE buffer followed by two washing solutions, one washing solution containing the 70-80% ethanol solution with 200 Mgcl2 and a second washing solution containing the only 50-80% of ethanol and the preferred elution buffer solution is Nuclease free water.

### Industrial Flowchart or Process Diagram of Large-Scale Purification of Nucleic Acid Extraction

FIG. 8 depicts an industrial method of extraction and purification of nucleic acid and other biological macromolecules from a sample. In this Industrial process flowchart or process diagram of large-scale purification of Nucleic Acid extraction is described in detail. An alkali cottonized hemp matrix column [102] is used for collecting or capturing a large amount of sample by loading the lysed or raw sample onto the system. The raw sample or biological sample is prepared inside a temperature, humidity, and pressure-controlled bio incubator mixer [106]. Different pressure pumps and regulators (P1, P2, P3, P4, . . . ) control the flow rate of the raw sample or biological samples as well as the lysate reagents from the individual, autoclavable reagent storage tanks [104]. Some of them may be dispensed at a desirable temperature, humidity, and controlled pressure. Every reagent and sample can only flow through closed steel pipes operated at each junction with individual 2-way, 3-way, 4-way, N numbers of way valves [108] as per branching. Some of the piping may require pressure, -temperature, and -humidity control while pumping the sample or reagents. The alkali cottonised hemp matrix column [102] is placed inside the matrix column chamber [110] which is fully autoclavable, temperature, humidity, and pressure controlled. This matrix column chamber can be further connected with different cleaning and processing reagent chambers [112] with pumping (P6, P7, P8, P9, P10, . . . . Pn) and piping, which helps to further cleaning, sample purification, and extraction process. Specific purified RNA and DNA extraction methods may involve several steps. A temperature, pressure, humidity humidity-controlled fully automated mixer chamber [114] can be placed or connected just after the matrix column chamber [110] which can further process the nucleic acid samples to incubate and overnight processing. The temperature, pressure, humidity humidity-controlled fully automated mixer chamber [114] may be connected with reagent chambers which are further connected with pumping (P12, P13, . . . . Pn) and piping, which helps to purify sample purification and extraction process. The sample may pass through some solid-state purification column [116] for ultrapurification and then finally be stored in a closed inert vial or purified sample container [118]. Every sample processing chamber can be autoclaved and an inert gas supply is provided as well and wastes are collected in different waste chambers. The alkali cottonised hemp matrix column as well as the purification column can be replaced easily when the quality of the final output is not optimal.

While specific language has been used to describe the disclosure, any limitations arising on account of the same are not intended. As would be apparent to a person in the art, various working modifications may be made to the method to implement the inventive concept as taught herein. The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

We claim:

1. A method for isolation of pure nucleic acid from a biological sample, method comprising steps—
a. loading the lysate sample to a column containing a matrix; and
b. washing and eluting the sample-bound matrix,
wherein the matrix is an alkali-cottonized hemp matrix.

2. The method as claimed in claim 1, wherein the alkali-cottonized hemp matrix is obtained by treating the cottonized hemp with a concentration ranging from about 1M-13M and 0.05 M to 7M for monovalent and divalent alkali respectively.

3. The method as claimed in claim 2, wherein monovalent and divalent alkali treatment of the cottonized hemp is carried out sequentially in an arbitrary combination thereof.

4. The method as claimed in claim 1, wherein the lysate sample is prepared by adding a lysis buffer, where the lysis buffer comprises a chelating agent, Tris buffer, detergent, and proteinase K, preferably 7-12% SDS with Tris EDTA 100 mM-200 mM (TE) and at a temperature range 40-60° Celsius.

5. The method as claimed in claim 1, wherein the washing step comprises a first wash of 50-90 percent water-soluble alcohol preferably ethanol, salts, and chelating agent and a second wash comprises a water-soluble alcohol preferably ethanol ranging from 50-90 (v/v) % and optimally 70% ethanol.

6. The method as claimed in claim 5, wherein the salts are monovalent or divalent ions, preferably MgCl₂ with concentrations ranging from 0.1-2M preferably 200 mM and 500 mM, and the chelating agent with concentrations ranging from 0.2M-1M preferably 0.2 M and 0.8 M.

7. The method as claimed in claim 1, wherein the elution for the monovalent matrix is carried out at 40 to 60° C. and by an agent selected from a group comprising water, distilled water, diethyl pyrocarbonate treated water, Milli-Q water, reverse osmosis purified water (RO water), Nuclease free water.

8. The method as claimed in claim 1, wherein the elution for the divalent matrix is carried out at 60° C. to 95° C. preferably at a temperature of 80° C., and is carried by an agent selected from a group comprising water, distilled water, diethyl pyrocarbonate treated water, Milli-Q water, reverse osmosis purified water (RO water), nuclease-free water with any chelating agent and Tris Buffer.

9. The method as claimed in claim 1, wherein the samples are preheated and maintained at a pH of 5-14, preferably pH 6-10.

10. The method as claimed in claim 1, wherein the alkali-cottonized matrix is obtained by
treating cottonized hemp fibers with a solution containing 0.1M-13M monovalent alkali or 0.05-7M divalent alkali in deionized water, followed by drying the hemp fibers at a temperature ranging from 20 to 70° C. for a duration of 30 minutes to 48 hours.
washing the hemp fibers 5-6 times, wherein the pH of the fiber is maintained at neutral pH;
drying the hemp fibers for 30 min-48 hrs at 40-60° C., and shredding and felting the hemp fibers.

11. The method as claimed in claim 1, wherein the washing step is followed by air drying.

12. The method as claimed in claim 1, wherein the shredding separates the sticky/clogged hemp fiber into pieces with optimum sizes and increases the binding efficiency.

13. The method as claimed in claim 1, wherein the felting interlocks the fibers and increases the binding efficiency.

14. An industrial system of extraction and purification of biological macromolecules from a sample, comprising:
a. Autoclavable reagent storage tanks [104] configured to collect lysed/raw samples, wherein the said incubator bio-mixer [104] is temperature-, humidity-, and pressure-controlled;
b. Incubator bio-mixer [106] configured to receive sample and lysate reagent via pressure pumps and regulators (P1, P2, P3, P4, . . . ) at a controlled flow rate from the individual, autoclavable reagent storage tanks [104];
c. Matrix column chamber [110] comprising HEMP matrix column [102] connected to a Processing reagent chambers [112] comprising alkalization chambers configured to treat the cottonized hemp fiber with 5-50% alkali solution in deionized water followed by drying the hemp fibers for 30 min-48 hrs at 20-70° C., via pumps (P6, P7, P8, P9, P10, . . . Pn);
d. Felting and Shredding Units configured to separate clogged cottonized hemp fibers into separate fibrous structures and interlocking of cottonized hemp fibers respectively.
e. Automated mixer chamber [114] placed downstream of the matrix column chamber [110] configured to incubate and processing of samples, wherein the automated mixer chamber [114] is connected with reagent chambers via pumping (P12, P13, . . . Pn) for sample purification and extraction process;
f. Optionally a solid-state purification column [116] for ultra purification; and
g. Optionally Closed inert vials or sterilized sample containers [118] for storage of the processed samples.

15. The system as claimed in claim 10, comprising washing chambers and drying chambers, wherein the washing chambers are configured to wash hemp fibers 5-6 times, wherein the pH of the fiber is maintained at 6-7 and the drying chambers are configured to completely dry the cottonized hemp fibers.

16. A method of obtaining an alkali-cottonized matrix by the system as claimed in claim 13, comprising
treating cottonized hemp fibers with a solution containing 0.1M-13M monovalent alkali or 0.05-7M divalent alkali in deionized water, followed by drying the hemp fibers at a temperature ranging from 20 to 70° C. for a duration of 30 minutes to 48 hours;
conveying the alkalized hemp to the washing chamber configured to wash the hemp fibers 5-6 times, wherein the pH of the fiber is maintained at neutral pH;
conveying the washed hemp to the drying chamber and to completely dry the fibers until fully dry and moisture-free; and
conveying the moisture-free alkali-cottonized hemp to the shredding and felting section.
